(19) United States
(12) Patent Application Publication
(10) Pub. No.: US 2013/0022635 A1
(43) Pub. Date: Jan. 24, 2013

(54) Expression Modulator For Clock Gene Bmal

(71) Applicants: Yoko Gozu, Yokohama-shi (JP); Shinichiro Haze, Yokohama-shi (JP); Keiko Mori, Yokohama-shi (JP); Kenichi Umishio, Yokohama-shi (JP)

(72) Inventors: Yoko Gozu, Yokohama-shi (JP); Shinichiro Haze, Yokohama-shi (JP); Keiko Mori, Yokohama-shi (JP); Kenichi Umishio, Yokohama-shi (JP)

(73) Assignee: SHISEIDO COMPANY, LTD., Chuo-ku, Tokyo (JP)

(21) Appl. No.: 13/638,620

(22) PCT Filed: Mar. 30, 2011
(86) PCT No.: PCT/JP2011/001940

§ 371 Date: Oct. 1, 2012

(30) Foreign Application Priority Data
Mar. 31, 2010 (JP) 2010-083016

(51) Int. Cl. A61K 36/14; A61K 36/185; A61K 36/758; A61K 36/53; A61K 36/28; A61K 36/324; A61K 36/899; A61K 36/738; A61K 36/81; A61K 36/05; A61K 36/61

(52) U.S. Cl. 424/195.17; 424/770; 424/725; 424/771; 424/745; 424/742; 424/750; 424/765; 424/760; 424/764

(57) ABSTRACT

The present invention provides a substance which can modulate expression of a Bmal gene effectively and with a wide range of application. Provided is an expression modulator for the Bmal gene comprising, as an active ingredient, one or more selected from the group consisting of hinoki cypress extract, chlorella extract, hop extract, Zanthoxylum extract, juniper oil, lavender oil, eucalyptus oil, olibanum, cypress oil, palmarosa oil, pineneedle oil, rose oil, ylang-ylang oil, elemi oil, petitgrain oil, pepper oil, thyme oil and chamomile oil.

### FIELD OF THE INVENTION

The present invention relates to an expression modulator for clock gene and more specifically to an expression modulator for clock gene Bmal and a circadian rhythm modulator comprising it.

### DESCRIPTION OF THE RELATED ART

Almost all living organisms on the earth have an internal “biological clock” which oscillates autonomously with a cycle of approximately 24 hours. The biological clock causes a biological daily fluctuation called circadian rhythm, which is considered to control diurnal changes of various biological phenomena (activities) including not only sleep-awake cycles of the organisms, but also body temperature, blood pressure, hormonal secretion, metabolism, as well as mental and physical activities, eating and the like. In recent years, it has been pointed out that the disturbance of the circadian rhythm is a pathogenic factor of various psychosomatic symptoms or disease conditions including sleep disorder, skin diseases, lifestyle-related diseases and neuropsychiatric disorders such as depression and the like.

As illustrated in FIG. 1, the biological clock is controlled by a rhythm-generating system comprising a group of genes called “clock genes.” In mammals, the core of the molecular mechanism of the clock is a feedback loop composed of the transcriptional promotion/suppression of the genes coding for four proteins, CLOCK, BMAL1, PERIOD and CRYPTOCHROME. The circadian rhythm is generated by pulsation of the feedback loop with a cycle of approximately 24 hours.

While the control center (central clock) of the circadian rhythm is located in the suprachiasmatic nucleus in the hypothalamus, it has become clear that the clock genes are expressed even in liver, kidney, skin and other peripheral tissues where the circadian rhythm is generated by a similar system. The expression of the peripheral clock genes is regulated by signals from the suprachiasmatic nucleus. It has further been established that the expression of the clock genes in the peripheral tissues and cells is controlled directly by signal stimulating factors like glucocorticoids, catecholamine, angiotensin II and others, to generate a physiological rhythm. In recent years, the circadian rhythm of cultured cells such as fibroblastic cells has been examined by inducing the circadian rhythm of the clock gene expression in vitro to synchronize the expression rhythm with the stimulating factors like glucocorticoids, forskolin, serum and the others, and by evaluating the circadian rhythm using their expression as the criteria (Non-patent Documents 1, 2 and 3).

The clock genes directly control expression rhythms of other genes as transcription factors, and indirectly control diurnal expression of many more genes through regulation of hormonal secretion and the like. It has become clear that disruption of expression rhythms of clock genes in organisms causes troubles of body organs or the endocrine system, leading to various diseases including lifestyle-related diseases such as hypertension (Non-patent Document 4). For example, a person with obesity has been reported to show abnormal expression of the clock genes, and associations with depression and cancer have also been reported. In addition, it has been elucidated that the clock genes regulate circadian rhythm of various physiological functions of the skin. In an experiment using normal human skin fibroblastic cells, the type I collagen gene was reported to be expressed in a circadian rhythm with an expression pattern similar to that of the clock gene Period 2 (Non-patent Document 5).

By modulating the expression of clock genes, it is possible to adjust various behavior rhythms and circadian rhythms of physiological functions of living organisms which are controlled by the clock genes. Accordingly, there is a strong need to develop an agent which can modulate the expression of the clock genes.

### [Non-patent Document 1]

H. Okamura, “Clock Genes in Cell Clocks: Roles, Actions, and Mysteries”, Journal of Biological Rhythms, Vol. 19, No. 5, pp. 388-399, 2004.

### [Non-patent Document 2]

A. Balsalobre et al., “A Serum Shock Induces Circadian Gene Expression in Mammalian Tissue Culture Cells”, Cell, Vol. 93, pp. 929-937, 1998.

### [Non-patent Document 3]

K. Yagita et al., “Molecular Mechanisms of the Biological Clock in Cultured Fibroblasts”, Science, Vol. 292, pp. 278-281, 2001.

### [Non-patent Document 4]

M. Hastings et al., “Circadian clocks: regulators of endocrine and metabolic rhythms”, Journal of Endocrinology, Vol. 195, pp. 187-198, 2007.

### [Non-patent Document 5]

K. Izumi et al., “Gaijitsu rizumu wo motsu hifuseiriidennshi no tansaku (Analysis of skin physiological genes having Circadian Rhythm expression)”, The Molecular Biology Society of Japan, 32nd Annual Meeting, Abstract 2P-0009, 2009.

### DISCLOSURE OF THE INVENTION

In view of the above-described circumstances, the object of the present invention is to provide an agent which can modulate the expression of a Bmal gene, which is a core gene of biological clocks.

The inventors have come to achieve the present invention as they have found that certain plant extracts or essential oils have a property to induce expression rhythms of clock gene Bmal and also to promote its expression.

An expression modulator for a Bmal gene of the present invention comprises, as an active ingredient, one or more selected from the group consisting of hinoki cypress extract, chlorella extract, hop extract, Zanthoxylum extract, juniper oil, lavender oil, eucalyptus oil, olibanum, cypress oil, palmarosa oil, pineneedle oil, rose oil, ylang-ylang oil, elemi oil, petitgrain oil, pepper oil, thyme oil and chamomile oil. It had heretofore not been known at all that the above-mentioned specific plant extract or essential oil can modulate the expression of the clock gene. In the present invention, the modulation of gene expression includes not only promotion of gene expression but also modulation of the rhythm of gene expression (phase or cycle).

The circadian rhythm modulator of the present invention comprises the above-mentioned expression modulator for a Bmal gene. As described above, the clock genes directly or indirectly control the diurnal expression of various genes involved in the function of body organs and in the endocrine system. By modulating the expression of a Bmal gene, which is a core gene of the biological clock, it is possible to regulate various behavioral rhythms of the living organism and circadian rhythms of physiological functions that are under the control of the gene.

Furthermore, the inventors of the present invention have found in experiments using human skin fibroblastic cells that some of the substances with expression modulating activity on the Bmal gene as described above can also modulate the expression of the gene for hyaluronic acid synthetase (Has). Thus, it is considered that the expression modulator of the Bmal gene of the present invention can improve the skin function by regulating the circadian rhythm of the hyaluronic acid production in the skin and by augmenting its production.

The expression modulator of the Bmal gene of the present invention may be used alone or in combination with an expression modulator for other clock genes. For example, it may be used in combination with a modulator of expression of a Period gene, one of the other core clock genes. In the circadian rhythm generating system as illustrated in FIG. 1, BMAL1, the expression product of a Bmal gene, forms a hetero dimer with CLOCK to promote transcription of the Period gene, while the expressed PERIOD (PER) forms a hetero dimer with CRYPTOCHROME (CRY) to suppress BMAL and CLOCK activities, so that a feedback loop is created which oscillates with a period of 24 hours. In mammals, circadian rhythms of Bmal and Period genes beat with a phase shift of approximately 12 hours. Bmal expression increases in the night time, while Period expression increases in the daytime. It is therefore considered that the circadian rhythm may be modulated more efficiently by regulating the core loop of the biological clock through the regulated expression of both Bmal and Period genes.

All of the expression modulators for the Bmal gene of the present invention are herbal medicines or fragrant essences. They may be applied by various administration modes such as transdermal, oral and inhaled administrations and may be used in various embodiments including pharmaceuticals, quasi-drugs, cosmetics, foods, miscellaneous goods, clothes and others. It is possible to improve various psychosomatic or dermal symptoms or diseases as a result of the malfunction of the circadian rhythm, by effectively modulating the expression of the Bmal gene.

Furthermore, among the expression modulators for the Bmal gene of the present invention, those which regulate Has expression rhythm can promote its expression and are thus considered to improve skin function by modulating the circadian rhythm of hyaluronic acid production in skin as well as by augmenting the amount of its production.

### BRIEF DESCRIPTION OF THE DRAWINGS

FIG. 1 is a schematic diagram showing only the core loop of a circadian rhythm generating system by clock genes.

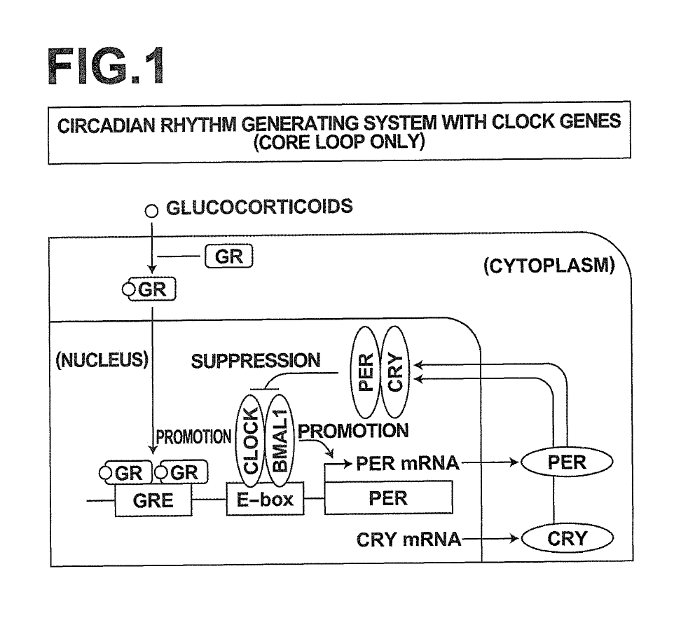

FIG. 2 is a graph showing induction of circadian rhythms of clock gene expression with cortisol and forskolin in cultured human skin fibroblastic cells.

FIG. 3 is a graph showing regulated expression of the clock gene Bmal1 by test substances in cultured human skin fibroblastic cells.

### BEST MODE FOR CARRYING OUT THE INVENTION

The expression modulator for the Bmal gene of the present invention comprises a certain plant extract or essential oil as its active ingredient.

The plant extracts which can modulate expression of the Bmal gene include a herbal medicine extract selected from the group consisting of hinoki cypress extract, chlorella extract, hop extract, and Zanthoxylum extract. As for the details of these herbal medicines, please refer to Japan Cosmetic Ingredients Dictionary (Nihon Hann-you Keshouhin Genryou Shu) fourth edition (YAKUJI NIPPO LIMITED).

The above-mentioned herbal medicine extracts may be obtained by commonly known techniques, for example, by immersing or heating to reflux the plant material from which each of the extracts is derived with an extraction solvent, followed by filtration and concentration. Any solvent which is normally used for extraction may be employed as the extraction solvent, including water, methanol, ethanol, propylene glycol, 1,3-butyleneglycol, glycerin and other alcohols, hydroalcoholic solvents, chloroform, dichloroethane, tetrachloromethane, acetone, ethylacetate, hexane and other organic solvents alone or in combination. The extracts obtained using the above-mentioned solvents may be used as they are, or alternatively, may be used after removing the impurities using an absorption technique with, for example, ion-exchange resin, or porous polymer (e.g. Amberlite XAD-2) column followed by elution with methanol or ethanol and then concentration. Extracts, for example, with water/ethylacetate and others may be used as well.

All of these herbal medicine extracts are commercially available and each of them is briefly described below.

Hinoki cypress extract is obtained from hinoki (Chamaecyparis obtusa) of the Cupressacea family, preferably extracted from the trunk (wood) of hinoki with 30% ethanol or the like.

Chlorella extract is obtained from the unicellular plant Chlorella vulgaris, preferably extracted from a Chlorella alga body with 80% ethanol or the like.

Hop extract is obtained from hop (Humulus lupulus) of the Cannabaceae family, preferably extracted from the female flower spike with 80% ethanol or the like.

Zanthoxylum extract is obtained from zanthoxylum fruit (Zanthoxylum piperitum), preferably extracted from the peel of Zanthoxylum fruit with 70% ethanol or the like.

Plant essential oils (fragrance) that can modulate expression of the Bmal gene include juniper oil, lavender oil, eucalyptus oil, olibanum, cypress oil, palmarosa oil, pineneedle oil, rose oil, ylang-ylang oil, elemi oil, petitgrain oil, pepper oil, thyme oil, and chamomile oil. These fragrances are all commercially available and will be briefly described below.

Juniper oil is an essential oil obtained by steam distillation of branches and leaves of juniper (Juniperus communis L), an evergreen tree of the Cupressacea family.

Lavender oil is an essential oil obtained by steam distillation of picked flower of Lavandula officinalis, a low shrub of the Lamiaceae family.

Eucalyptus oil is an essential oil obtained by steam distillation of branches and leaves of eucalyptus (e.g. Eucalypus globulus) an evergreen timber tree belonging to the Myrtaceae family.

Olibanum is an essential oil obtained by steam distillation of resin seeping out of bark of a shrub or a low timber tree such as Boswellia carterii Birdw, which belongs to the Burseraceae family of the Geraniales order.

Cypress oil is an essential oil obtained by steam distillation of branches and leaves of cypress (Cupressus sempervirene).

Palmarosa oil is an essential oil obtained by steam distillation of leaves of palmarosa (Cymbopogon martini) of the Poaceae family.

Pineneedle oil is an essential oil obtained by steam distillation of needle leaves and small branches of a pine tree such as Pinus sylvestris of the Pinaceae family or Sakhalin fir of the Abies genus.

Rose oil is an essential oil obtained by steam distillation of cabbage rose (Rosa centifolia L), Damask rose (Rosa damascena Mill.), and other rose flowers.

Ylang-ylang oil is an essential oil obtained by direct steam distillation of flowers of a plant Canning odorata belonging to the Anonaceae family or extraction of fresh flowers with petroleum ether or other solvents.

Elemi oil is an essential oil obtained by steam distillation of resin (oleoresin) seeped from the bark of elemi, a medium to tall tree of the Canarium order of the Burseraceae family.

Petitgrain oil is an essential oil obtained by steam distillation of leaves and other part of bitter orange (Citrus aurantium) of the Rutaceae family.

Pepper oil is an essential oil obtained by steam distillation of berries of pepper (Piper nigrum L.), a perennial plant belonging to the Piperaceae family.

Thyme oil is an essential oil obtained by steam distillation of whole plant of thyme (Thymus vulgaris L.), a perennial herb of the Lamiaceae family.

Chamomile oil is an essential oil obtained by steam distillation of chamomile (Ornemis mutticaulls).

The expression modulator for the Bmal gene of the present invention may comprise one or more of extracts (herbal medicines) or essential oils (fragrances) of the above-mentioned plants. For example, the expression modulator may comprise one or more of the above-mentioned herbal medicines in combination with one or more of the above-mentioned fragrances.

The modulator of circadian rhythm of the present invention contains one or more of the above-mentioned plant extracts or essential oils as an expression modulator for the Bmal gene.

The expression modulator for the Bmal gene may be used alone or in combination with an agent having an action to modulate expression of other clock genes.

Examples of the other clock genes include Period genes (Period1, Period2, Period3), Clock gene, Cryptochrome gene, albumin site D-binding protein (Dbp) gene, E4BP4 gene, Npas2 gene, and Rev-erb gene. It is, however, preferable for the expression modulator for the Bmal gene to be used in combination with an expression modulator for Period, Clock and/or Cryptochrome gene(s), other core genes of the biological clock, and especially preferable to use in combination with an expression modulator for a Period gene(s).

The expression modulators for the Period gene include, but are not limited to, herbal medicines such as arnica, nuphar, black tea extract, Zanthoxylum and other extracts; and fragrances such as juniper, cedar, lavender, clove bud, cypress, rose, ylang-ylang, galbanum, petitgrain, pepper, thyme, basil, beta-caryophyllene and other essential oils. It has been confirmed that these herbal medicines or fragrances can induce expression rhythm of a Period gene or promote its expression in cultured skin fibroblastic cells.

Further, the expression modulator for the Bmal gene and the circadian rhythm modulator of the present invention may be used alone or may be contained in various substances. Depending on the kind of substance, any constituent may supplementarily be included as well as the above-mentioned indispensable ingredient.

For example, when the substance is an external formulation to be applied on the skin, any ingredient which is normally found in such an external formulation may be contained together with the above-mentioned expression modulator for the Bmal gene depending on its dosage form (e.g. liquid formulation, powder formulation, granular powder formulation, aerosolized formulation, solid formulation, gel formulation, patch formulation, suppository formulation, and others) or its product form (e.g. cosmetics, pharmaceuticals, quasi-drugs, and others). An external formulation to be applied on the skin encompasses compositions to be applied on the skin (including head skin, head hair and nails) in general and includes cosmetics such as skin care products, make-up products, hair care products, face wash products, hair wash products and others, as well as various pharmaceuticals and quasi-drugs such as ointment formulations, patch formulations, suppository formulations, tooth pastes and others. The dosage forms include, but are not limited to, water-based systems, solubilized systems, emulsions, oil-based systems, gels, pastes, ointments, aerosols, water-oil two-phase systems, water-oil-powder three phase systems, and others. When the external formulation to be applied on the skin is a cosmetic, it includes perfumes, eaux de toilet, eaux de cologne, creams, emulsions, foundations, face powders, lip sticks, soaps, shampoos and conditioners, body shampoos, body rinses, body powders, bath soaps, and others.

The expression modulator for the Bmal gene of the present invention may be contained in air fresheners, deodorants, aromatic candles, incenses, stationaries, purses, bags, shoes, and any other miscellaneous goods; underwear, outfits, hats, pairs of stockings, socks and any other clothes; or as a food supplement in powders, granules, capsules, and a variety of other formulations; and snacks, drinks, and any other food. Furthermore, when the expression modulator for the Bmal gene of the present invention contains the above-mentioned essential oil (fragrance), it may be used in an inhalant such as a pharmaceutical inhalation product and an atomizing agent, as long as the present invention produces its effect.

The embodiments of the expression modulator for the Bmal gene of the present invention are exemplarily illustrated in this specification. The present invention, however, is not limited to the embodiments described in this specification, but may be adopted in any mode of use, under conditions that produce the effects of the present invention. Together with the expression modulator for the Bmal gene of the present invention, other agents having the action to modulate circadian rhythm may be combined depending on the specific mode of use, under conditions that do not impede the effects of the present invention.

The content of the expression modulator for the Bmal gene of the present invention in a substance is not particularly limited, and may be selected appropriately according to the type and form of the herbal medicine or fragrance used, the substance, and the like, but is for example 0.00001 mass % to 100 mass %, preferably 0.0001 mass % to 50 mass %, and more preferably 0.0001 mass % to 20 mass % of the total mass of the substance.

The specific application of the expression modulator for the Bmal gene or the modulator of circadian rhythm of the present invention as well as the substance comprising the modulator is not particularly limited as long as it pertains to the modulation of circadian rhythms. For example, it can be applied to the prevention, improvement, treatment or the like of jet lag syndrome, shift work syndrome, delayed sleep phase syndrome, non-24-hour sleep-wake disorder, depression with circadian rhythm sleep disorder and the like, as well as insomnia, poor physical conditions, attention deficit, apathy, rough skin and various other symptoms that are associated with the disturbance of circadian rhythm.

### EXAMPLES

The present invention will be described in detail below with examples, but the present invention is not limited to the examples. Skin fibroblastic cells, epithelial cells, endothelial cells, pigment cells, fat cells, nerve cells and various other types of cells may be used as culture cells. In the examples, however, evaluations were carried out with human skin fibroblastic cells. Because the core system of the clock gene is common to all species of organisms and all types of cells, it is thought that evaluation results from human skin fibroblastic cells should be applicable to other species of organisms and other types of cells. In humans, two genes are known as Bmal genes; Bmal1 and Bmal2. They are thought to behave similarly as they belong to the same gene family. In the examples below, Email expression was determined as a representative.

### Examination of Evaluation System for Clock Gene Expression Rhythm Using Cultured Human Skin Fibroblastic Cells

It was confirmed that clock gene expression rhythm can be evaluated in a system using cultured human skin fibroblastic cells.

As the cultured human fibroblastic cells, fibroblastic cells from normal human skin were purchased (Cell Application, Inc.) and used in the experiments. They were inoculated in DMEM medium supplemented with 10% FBS, 20 mM HEPES, Glutamax and antibacterial agents and cultured at 37° C. in 5% CO₂. On the 6th day of culture, 50 ng/mL of cortisol or 10 μM of forskolin was added, and the samples were harvested at various times after time 0, which was defined as the time immediately after the addition of cortisol or forskolin. RNA was extracted from the cells with a commercially available RNA extraction kit, and the amounts of expression of the intended genes were measured by RT-PCR technology using commercially available PCR primers (Perfect Real Time Primer, Takara Bio). As for clock genes, amounts of expression of Period (represented by Period1) and Bmal1, which are involved in the core system, were determined. Similarly, the amounts of expression of a housekeeping gene RPLP0 were quantified and used as an internal standard to calculate the relative expression of the intended genes to RPLP0.

In living organisms, glucocorticoids such as cortisol are involved in regulating the biological clock in peripheral tissue and the like, and it is thought that blood concentration of cortisol rises when waking in the morning, to reset the biological clock. In cultured cells, individual cells usually keep their rhythm independently. The expression rhythms of clock genes can be synchronized to induce a circadian rhythm by stimulating with a signal stimulation factor such as cortisol or forskolin.

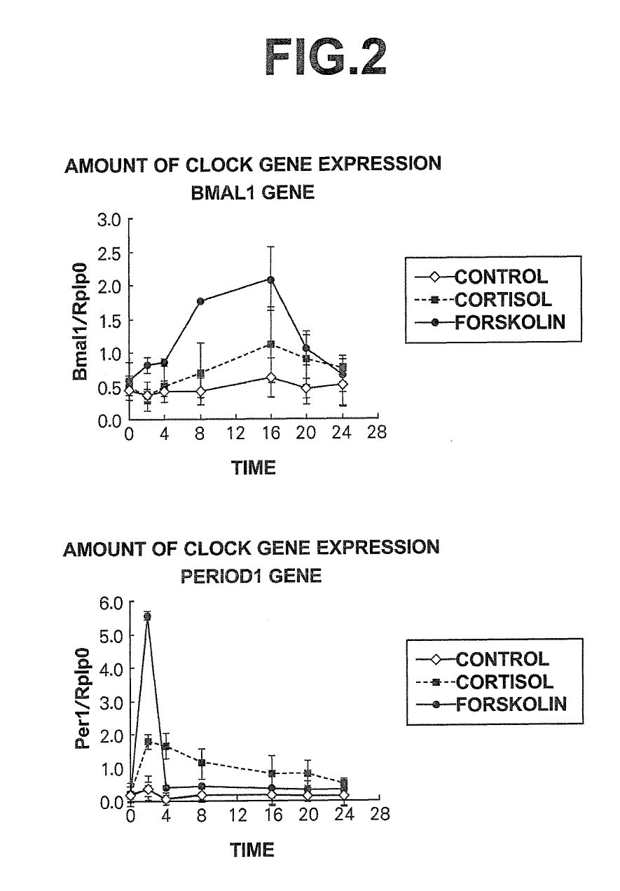

The results are shown in FIG. 2. It was confirmed that expression of Bmal1 and Period1 peaks at about 16 hours and about 2 hours, respectively, after stimulation, and that both genes are expressed recurrently with a circadian rhythm of about a 24 hour cycle.

### Evaluation of Test Substances on the Expression Modulating Effect for a Bmal Gene

The above-mentioned results of the evaluation system using cultured human skin fibroblastic cells demonstrate that Bmal1 shows a circadian rhythm with a peak of gene expression at about 16 hours after the stimulation with a reagent. Test substances were evaluated for the expression modulating effect on a Bmal gene based on the amount of gene expression at 16 hours after stimulation. To confirm the induction of expression rhythm for the Bmal gene, the amount of expression of the Bmal gene at 2 hours after stimulation was also determined.

Fibroblastic cells from normal human skin (Cell Application, Inc.) were inoculated according to a method similar to those used above. Each test substance was added on the 6th day of culture to a final concentration of 100 ppm, and the cells were harvested 2 and 16 hours after stimulation. RNA was extracted from the cells with a commercially available RNA extraction kit. The amounts of expression of the Bmal gene were determined by RT-PCR technology using commercially available PCR primers (Perfect Real Time Primer, Takara Bio).

Furthermore, gene expression was also similarly determined for hyaluronic acid synthetase (HAS) involved in the production of hyaluronic acid which plays an important role in retaining skin moisture. HAS1, HAS2 and HAS3 are known as HAS, and HAS2 was examined as a representative hyaluronic acid synthetase.

The amounts of gene expression of a housekeeping gene RPLP0 were quantified and used as an internal standard to calculate the relative expression of the target genes with respect to RPLP0. Dunnett's multiple comparison test was performed on obtained measurements, and measurements with a significance level of a one-sided 5% compared with the control were deemed to be significantly effective.

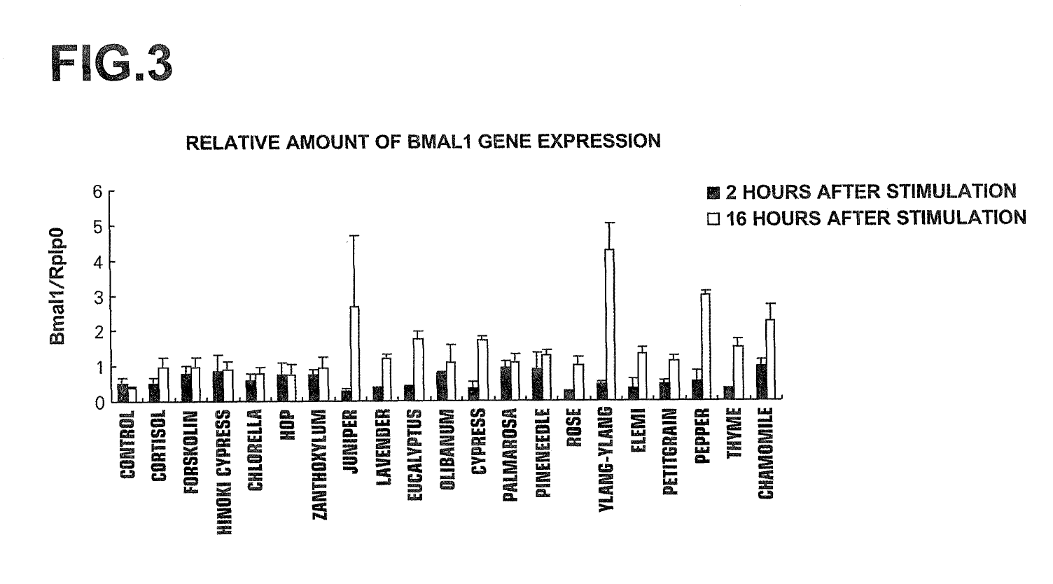

FIG. 3 shows relative amounts of gene expression of the Small gene at 2 and 16 hours after addition of a variety of test substances and cortisol or forskolin as positive controls.

Table 1 below shows relative amounts of gene expression of the Bmal1 gene at 16 hours. For some test substances, relative amounts of gene expression of the Has2 gene were also shown in Table 1.

TABLE 1

| | Relative amount of | Relative amount of Has2 |
|---|---|---|
| | Bmal1 gene expression | gene expression |
| Test substance | (16 hours) | (16 hours) |
| Control | 0.41 | 4.6 |

Herbal medicine

| | |
|---|---|
| Hinoki Cypress | 0.64** |
| Chlorella | 0.63** |
| Hop | 0.55* |
| Zanthoxylum | 0.6** |

Fragrance

| | | |
|---|---|---|
| Juniper | 2.69** | 163 |
| Lavender | 1.2** | |
| Eucalyptus | 1.73** | 1549** |
| Olibanum | 1.09* | |
| Cypress | 1.13** | 62.6** |
| Palmarosa | 1.07* | |
| Pineneedle | 1.26** | |
| Rose | 1.01* | |
| Ylang-ylang | 4.25** | 66** |
| Elemi | 1.3** | 448** |
| Petitgrain | 1.13* | |
| Pepper | 1.98** | 78.8** |
| Thyme | 1.5** | |
| Chamomile | 2.25** | |

*p < 0.05,
**p < 0.01

It was demonstrated that hinoki cypress extract, chlorella extract, hop extract, Zanthoxylum extract, juniper oil, lavender oil, eucalyptus oil, olibanum, cypress oil, palmarosa oil, pineneedle oil, rose oil, ylang-ylang oil, elemi oil, petitgrain oil, pepper oil, thyme oil, and chamomile oil, as well as positive controls of cortisol and forskolin, can induce an expression rhythm with a peak at 16 hours after stimulation, and significantly increase amounts of gene expression of Bmal1 and, accordingly, that these herbal medicines or fragrances can modulate expression of the Bmal gene.

In addition, it was suggested that juniper, eucalyptus, cypress, ylang-ylang, elemi and pepper can improve or augment skin functions by enhancing hyaluronic acid production, as they were shown to promote Has2 gene expression 16 hours after stimulation.

The expression modulator for the Bmal gene of the present invention may be used in combination with an expression modulator for a Period gene, which include, but are not limited to, herbal medicines such as arnica, nuphar, black tea extract, Zanthoxylum, and other extracts; and fragrances such as juniper, cedar, lavender, clove bud, cypress, rose, ylang-ylang, galbanum, petitgrain, pepper, thyme, basil, beta-caryophyllene, and other oils. It has been confirmed that these herbal medicines or fragrances can induce expression rhythm of the

Period1 gene or promote its expression, as a result of examining expression rhythm of Period genes in cultured skin fibroblastic cells with Period1 as the representative. Table 2 below shows relative amounts of gene expression of the Period1 gene at 16 hours after stimulation.

TABLE 2

| | Relative amount of |
|---|---|
| | Period1 gene expression |
| Test substance | (2 hours) |
| Control | 0.23 |

Herbal medicine

| | |
|---|---|
| Arnica | 0.56** |
| Black tea extract | 0.48* |
| Nuphar | 0.51** |
| Zanthoxylum | 0.42* |

Fragrance

| | |
|---|---|
| Juniper | 0.67* |
| Cedar | 0.7* |
| Lavender | 0.88** |
| Clove Bud | 1.11** |
| Cypress | 1.29** |
| Rose | 0.75* |
| Ylang-ylang | 1.08** |
| Galbanum | 0.76* |
| Petitgrain | 1.06** |
| Pepper | 1.44** |
| Thyme | 1.81** |
| Basil | 0.68* |
| beta-Caryophyllene | 0.44* |

*p < 0.05,
**p < 0.01

### COMPOSITIONAL EXAMPLES

Compositional examples of the expression modulator for the Bmal gene of the present invention are given below, but the present invention is not limited to the following. In the compositional examples below, ylang-ylang oil is used as the expression modulator for the Bmal gene of the present invention. One, or a mixture of more than one, of the herbal medicines and/or fragrances which are described above as being capable of modulating the expression of the Bmal gene may be contained. Compositional amounts are all represented by mass percent relative to the total amount of each product.

### [Fragrance]

| | | |
|---|---|---|
| (1) | Alcohol | 75.0 |
| (2) | Purified water | remainder |
| (3) | Dipropylene glycol | 5.0 |
| (4) | Expression Modulator for Bmal Gene of the Present | 10.0 |
| | Invention: ylang-ylang oil | |
| (5) | Antioxidant | 8.0 |
| (6) | Dye | as needed |
| (7) | UV absorbent | as needed |

### [Room Freshener]

| | | |
|---|---|---|
| (1) | Alcohol | 80.0 |
| (2) | Purified water | remainder |
| (3) | Antioxidant | 5.0 |
| (4) | Expression modulator for Bmal gene of the present | 3.0 |
| | invention (ylang-ylang oil) | |
| (5) | 3-methyl-3-methoxybutanol | 5.0 |
| (6) | Dibenzylidene sorbitol | 5.0 |

### [Incense]

| | | |
|---|---|---|
| (1) | Makko powder | 75.5 |
| (2) | Sodium benzoate | 15.5 |
| (3) | Expression modulator for Bmal gene of the present | 5.0 |
| | invention (ylang-ylang oil) | |
| (4) | Eucalyptus oil | 1.0 |
| (5) | Purified water | remainder |

### [Bath Salts]

| | | |
|---|---|---|
| (1) | Sodium sulfate | 45.0 |
| (2) | Sodium bicarbonate | 45.0 |
| (3) | Lavender oil | 9.0 |
| (4) | Expression modulator for Bmal gene of the present invention | 1.0 |
| | (ylang-ylang oil) | |

### [Massage Gel]

| | | |
|---|---|---|
| (1) | Erythritol | 2.0 |
| (2) | Caffeine | 5.0 |
| (3) | Phellodendron amurense bark extract | 3.0 |
| (4) | Glycerin | 50.0 |
| (5) | Carboxyvinyl polymer | 0.4 |
| (6) | Polyethylene glycol 400 | 30.0 |
| (7) | Trisodium edetate | 0.1 |
| (8) | Polyoxylene (10) methylpolysiloxane copolymer | 2.0 |
| (9) | Squalane | 1.0 |
| (10) | Potassium hydroxide | 0.15 |
| (11) | Expression modulator for Bmal gene of the | 1.0 |
| | present invention (ylang-ylang oil) | |

### [Massage Cream]

| | | |
|---|---|---|
| (1) | Solid paraffin | 5.0 |
| (2) | Beeswax | 10.0 |
| (3) | Vaseline | 15.0 |
| (4) | Fluid paraffin | 41.0 |
| (5) | 1,3-butylene glycol | 4.0 |
| (6) | Glycerin monostearate | 2.0 |
| (7) | POE (20) sorbitan monolaurate ester | 2.0 |
| (8) | Borax | 0.2 |
| (9) | Caffeine | 2.0 |
| (10) | Preservative | as needed |
| (11) | Antioxidant | as needed |
| (12) | Expression modulator for Bmal gene of the present | 1.0 |
| | invention (ylang-ylang oil) | |
| (13) | Purified water | remainder |

### [Aromatic Fiber]

Microcapsules containing the expression modulator for Bmal gene of the present invention (particle diameter: no greater than 50 μm (micrometer); percentage of essential oil in microcapsule: 50 wt %) were added to a cuproammonium cellulose solution (cellulose concentration: 10 wt %; ammonium concentration: 7 wt %; copper concentration 3.6 wt %) in the range of 0.1 wt % to 20 wt % of the cellulose weight, mixed, and spun by a conventional wet spinning method, and aromatic fiber was obtained following a refining step and a drying step.

### [Granules]

| | | |
|---|---|---|
| (1) | Sucralose | 0.1 |
| (2) | Expression modulator for Bmal gene of the present | 0.1 |
| | invention (ylang-ylang oil) | |
| (3) | Flavoring | 5.0 |
| (4) | Excipient (Ceolus) | 10.0 |
| (5) | Maltitol | remainder |

### [Tablets (Chewable Type)]

| | | |
|---|---|---|
| (1) | Inositol | 11.0 |
| (2) | Maltitol | 21.0 |
| (3) | Sucrose | 0.5 |
| (4) | Salmon sperm extract (DNA Na) | 0.1 |
| (5) | Yeast extract | 0.1 |
| (6) | Expression modulator for Bmal gene of the present | 0.1 |
| | invention (ylang-ylang oil) | |
| (7) | Flavoring | 5.0 |
| (8) | Excipient | remainder |

### [Tablets]

| | | |
|---|---|---|
| (1) | Lubricant (sucrose fatty acid ester, etc.) | 1.0 |
| (2) | Gum arabic aqueous solution (5%) | 2.0 |
| (3) | Acidulant | 1.0 |
| (4) | Colorant | as needed |
| (5) | Expression modulator for Bmal gene of the present | 0.1 |
| | invention (ylang-ylang oil) | |
| (6) | Sugars (powdered sugar, sorbitol, etc.) | remainder |

### [Candy]

| | | |
|---|---|---|
| (1) | Sugar | 50.0 |
| (2) | Starch syrup | 47.95 |
| (3) | Organic acids | 2.0 |
| (4) | Expression modulator for Bmal gene of the present invention | 0.05 |
| | (ylang-ylang oil) | |

### [Gum]

| | | |
|---|---|---|
| (1) | Sugar | 43.0 |
| (2) | Gum base | 30.95 |
| (3) | Glucose | 10.0 |
| (4) | Starch syrup | 16.0 |
| (5) | Expression modulator for Bmal gene of the present invention | 0.05 |
| | (ylang-ylang oil) | |

Products of these compositional examples can regulate expression of Bmal gene and modulate the circadian rhythm of the living organism by a trial use of each product form in a typical manner of use.

1. A modulator for circadian rhythm comprising, as an active ingredient, one or more selected from the group consisting of hinoki cypress extract, chlorella extract, hop extract, Zanthoxylum extract, juniper oil, lavender oil, eucalyptus oil, olibanum, cypress oil, palmarosa oil, pineneedle oil, rose oil, ylang-ylang oil, elemi oil, petitgrain oil, pepper oil, thyme oil and chamomile oil.

2. An expression modulator for the Bmal gene comprising, as an active ingredient, one or more selected from the group consisting of hinoki cypress extract, chlorella extract, hop extract, Zanthoxylum extract, juniper oil, lavender oil, eucalyptus oil, olibanum, cypress oil, palmarosa oil, pineneedle oil, rose oil, ylang-ylang oil, elemi oil, petitgrain oil, pepper oil, thyme oil and chamomile oil.

3. The expression modulator for the Bmal gene according to claim 2, wherein the expression modulator is used in combination with an expression modulator for a Period gene.
